(19) United States
(12) Patent Application Publication
(10) Pub. No.: US 2026/0256528 A1
(43) Pub. Date: Sep. 3, 2026

(54) ALARM SYSTEM FOR A SURGICAL ROBOT

(71) Applicant: CMR Surgical Limited, Cambridge (GB)

(72) Inventors: Edward James Wildin Tucker, Cambridge (GB); Johnathan Hung, Cambridge (GB); David Moore, Cambridge (GB)

(73) Assignee: CMR Surgical Limited, Cambridge (GB)

(21) Appl. No.: 18/995,637

(22) PCT Filed: Aug. 11, 2023
(86) PCT No.: PCT/GB2023/052128

§ 371 Date: Jan. 16, 2025

(30) Foreign Application Priority Data
Aug. 15, 2022 (GB) 2211909.3

(51) Int. Cl. A61B 34/30; A61B 34/20; A61B 90/00

(52) U.S. Cl. CPC A61B 34/30; A61B 34/20; A61B 90/03; A61B 90/37

(57) ABSTRACT

An alarm system is provided for a surgical robot. The surgical robot comprises a surgical robot arm and a surgical instrument attached to a distal end of the surgical robot arm. The surgical robot arm is driven using control signals which attempt to maintain an intersection between the surgical instrument and a pivot point. A shaft axis line which is coincident with an axis of a shaft of the instrument is determined. An indication of a retraction distance is determined which indicates how far the instrument is retracted relative to the pivot point. A perpendicular distance is determined between the shaft axis line and the pivot point in a direction perpendicular to the shaft axis line. The determined perpendicular distance is compared with an alarm threshold. An alarm is raised in response to determining that the determined perpendicular distance is greater than the alarm threshold. The alarm threshold is dependent upon the indicated retraction distance.

### BACKGROUND

This invention relates to an alarm system for a surgical robot.

It is known to use robots for assisting and performing surgery. A surgical robot may comprise a surgical robot arm and a surgical instrument attached to a distal end of the surgical robot arm. FIG. 1 illustrates an example surgical robotic system 100, which comprises a surgical robot arm 101 for manipulating tissue. The surgical robot arm 101 comprises a base 109. The base supports the surgical robot arm, and is itself attached rigidly to, for example, the operating theatre floor, the operating theatre ceiling or a trolley. The surgical robot arm 101 is articulated by means of multiple joints 104 along its length, which are used to locate a surgical instrument 106 in a desired location relative to a patient 102. The surgical instrument 106 could, for example, be a cutting or grasping device. A surgical instrument 106 is attached to the distal end of the surgical robot arm 101. The surgical instrument 106 is inserted into the body of the patient 102, e.g. via an access port 117, so as to access a surgical site within the body of the patient 102. At its distal end the surgical instrument comprises an end effector for performing aspects of a medical procedure. This type of medical procedure is often referred to as a minimally invasive surgical procedure.

The configuration of the surgical robot arm 101 may be remotely controlled in response to inputs received at a remote surgeon console 120. A surgeon may provide inputs to the remote console. The remote surgeon console may comprise one or more surgeon input devices 123. For example, these may take the form of one or more hand controllers, foot pedals, interactive touch screens etc. A video feed of the surgical site may be captured by an endoscope, often attached to a further surgical robot arm (not shown in FIG. 1 for simplicity), and displayed at a display 121 of the remote surgeon console.

A control system 124 connects the surgeon console 120 and the surgical robot arm 101. The control system 124 receives inputs from the surgeon input device(s) 123 and converts those inputs to control signals for controlling the surgical robot arm 101.

An alarm may be raised in some situations. For example, if the surgical robotic system 100 is not working correctly or if there may be a risk to patient safety then an alarm may be raised. There are many different types of alarms which may be raised in a surgical robotic system during a surgical procedure, and the alarms may have different priorities, e.g. there may be low priority alarms, medium priority alarms and high priority alarms. The surgical robotic system 100 may raise different alarms in different ways. For example, the surgical robotic system 100 may raise the alarm by doing one or more of the following: (i) switch on a warning light, (ii) output an audible alarm, (iii) disable one, some or all functions of the surgical robot, and (iv) change an operating mode of the surgical robot arm.

Raising an alarm causes a significant disruption to the workflow of the surgical procedure. Some functionality may be disabled and the surgical robotic system may need to be reset or retrained after an alarm has been raised before the workflow of the surgical procedure can continue. As such, avoiding raising an alarm in situations when it is not necessary for an alarm to be raised will improve the workflow of surgical procedures.

### SUMMARY

There is provided an alarm system for a surgical robot, the surgical robot comprising a surgical robot arm and a surgical instrument attached to a distal end of the surgical robot arm, wherein the surgical robot arm is driven using control signals which attempt to maintain an intersection between the surgical instrument and a pivot point, wherein the alarm system is configured to:
determine a shaft axis line which is coincident with an axis of a shaft of the instrument;
determine an indication of a retraction distance which indicates how far the instrument is retracted relative to the pivot point;
determine a perpendicular distance between the shaft axis line and the pivot point in a direction perpendicular to the shaft axis line; and
compare the determined perpendicular distance with an alarm threshold and raise an alarm in response to determining that the determined perpendicular distance is greater than the alarm threshold,
wherein the alarm threshold is dependent upon the indicated retraction distance.

The alarm system may be configured to determine the indication of the retraction distance by determining a distance along the shaft axis line between a predetermined position on the shaft axis line and the point on the shaft axis line which is closest to the pivot point.

The surgical robot arm may comprise a series of joints by which its configuration can be altered. The predetermined position on the shaft axis line may be at the most distal joint of the joints which control the orientation of the shaft axis line.

The alarm threshold may be a function of the determined indication of the retraction distance. The alarm threshold may have two regimes: (i) a first regime for a first range of retraction distances in which the tip of the instrument is either at least as far along the shaft axis line as the pivot point or less far along the shaft axis line than the pivot point by an amount which is not greater than a predetermined amount, and (ii) a second regime for a second range of retraction distances in which the tip of the instrument is less far along the shaft axis than the pivot point by an amount which is greater than the predetermined amount. The predetermined amount may be 4 cm.

The alarm threshold may have a constant value (e.g. 0.025 m) in the first regime.

The alarm threshold may have a value in the second regime that increases as the indicated retraction distance increases.

The alarm threshold may have a value in the second regime that increases linearly as the indicated retraction distance increases.

The alarm threshold may be a continuous function of the determined indication of the retraction distance.

The alarm system may be configured to raise the alarm by doing one or more of the following: (i) switch on a warning light, (ii) output an audible alarm, (iii) disable one, some or all functions of the surgical robot, (iv) change an operating mode of the surgical robot arm, (v) cause one or more hand controllers to vibrate, and (vi) display an alarm sign.

The alarm system may be configured to not raise the alarm in response to determining that the perpendicular distance is less than the alarm threshold.

The pivot point may be determined using a port training process.

The alarm system may be further configured to compare the determined perpendicular distance with a clash threshold and raise a clash signal in response to determining that the determined perpendicular distance is greater than the clash threshold.

The clash threshold may be dependent upon the indicated retraction distance.

The clash threshold may be dependent upon an operating mode of the surgical robot arm.

The clash threshold may be less than the alarm threshold. In particular, the clash threshold may be less than the alarm threshold for all indicated retraction distances.

The clash threshold may be a function of the determined indication of the retraction distance. The clash threshold may have two regimes: (i) a first regime for a first range of retraction distances in which the tip of the instrument is either at least as far along the shaft axis line as the pivot point or less far along the shaft axis line than the pivot point by an amount which is not greater than a predetermined amount, and (ii) a second regime for a second range of retraction distances in which the tip of the instrument is less far along the shaft axis than the pivot point by an amount which is greater than the predetermined amount. The predetermined amount may be 4 cm.

The clash threshold may have a constant value (e.g. 0.015 m or 0.017 m) in the first regime.

The clash threshold may have a value in the second regime that increases as the indicated retraction distance increases.

The clash threshold may have a value in the second regime that increases linearly as the indicated retraction distance increases.

The clash threshold may be a continuous function of the determined indication of the retraction distance.

The alarm system may be configured to raise the clash signal by doing one or more of the following: (i) display a clash warning icon on a screen, (ii) output an audible warning, (iii) cause a hand controller of a surgeon console to vibrate, (iv) disable one, some or all functions of the surgical robot 300, and (v) change an operating mode of the surgical robot arm 301.

The alarm system may be configured to not raise the clash signal in response to determining that the perpendicular distance is less than the clash threshold.

There may be provided a surgical robotic system comprising:
a surgical robot comprising a surgical robot arm and a surgical instrument attached to a distal end of the surgical robot arm;
a surgeon input device for controlling the surgical robot; and
an alarm system as described herein.

There is provided a computer readable storage medium have stored thereon computer readable instructions that when executed on one or more processors cause a method of raising an alarm for a surgical robot to be performed, the surgical robot comprising a surgical robot arm and a surgical instrument attached to a distal end of the surgical robot arm, wherein the surgical robot arm is driven using control signals which attempt to maintain an intersection between the surgical instrument and a pivot point, wherein the method of raising an alarm comprises:
determining a shaft axis line which is coincident with an axis of a shaft of the instrument;
determining an indication of a retraction distance which indicates how far the instrument is retracted relative to the pivot point;
determining a perpendicular distance between the shaft axis line and the pivot point in a direction perpendicular to the shaft axis line;
comparing the determined perpendicular distance with an alarm threshold; and
raising an alarm in response to determining that the determined perpendicular distance is greater than the alarm threshold,
wherein the alarm threshold is dependent upon the indicated retraction distance.

There is provided a method of raising an alarm for a surgical robot, the surgical robot comprising a surgical robot arm and a surgical instrument attached to a distal end of the surgical robot arm, wherein the surgical robot arm is driven using control signals which attempt to maintain an intersection between the surgical instrument and a pivot point, wherein the method comprises:
determining a shaft axis line which is coincident with an axis of a shaft of the instrument;
determining an indication of a retraction distance which indicates how far the instrument is retracted relative to the pivot point;
determining a perpendicular distance between the shaft axis line and the pivot point in a direction perpendicular to the shaft axis line;
comparing the determined perpendicular distance with an alarm threshold; and
raising an alarm in response to determining that the determined perpendicular distance is greater than the alarm threshold,
wherein the alarm threshold is dependent upon the indicated retraction distance.

There may be provided a clash detection system for a surgical robot, the surgical robot comprising a surgical robot arm and a surgical instrument attached to a distal end of the surgical robot arm, wherein the surgical robot arm is driven using control signals which attempt to maintain an intersection between the surgical instrument and a pivot point, wherein the clash detection system may be configured to:
determine a shaft axis line which is coincident with an axis of a shaft of the instrument;
determine a perpendicular distance between the shaft axis line and the pivot point in a direction perpendicular to the shaft axis line; and
compare the determined perpendicular distance with a clash threshold and raise a clash signal in response to determining that the determined perpendicular distance is greater than the clash threshold.

The clash detection system may be further configured to determine an indication of a retraction distance which indicates how far the instrument is retracted relative to the pivot point. The clash threshold may be dependent upon the indicated retraction distance.

### BRIEF DESCRIPTION OF THE DRAWINGS

The present invention will now be described by way of example with reference to the accompanying drawings. In the drawings:

FIG. 1 shows an example surgical robotic system;

FIG. 2 shows an example surgical instrument;

FIG. 3 shows an example surgical robot;

FIG. 4 shows an example surgical robotic system;

FIG. 5 shows an example surgical robot arm calibration process;

FIG. 6 is a flow chart for a method of raising an alarm for a surgical robot;

FIG. 7a shows the relative positions of a wrist joint of the surgical robot arm, the surgical instrument and a pivot point when the surgical robotic system is operating normally;

FIG. 7b shows the relative positions of the wrist joint of the surgical robot arm, the surgical instrument and the pivot point in a first situation in which the surgical robotic system is not able to respect the pivot point;

FIG. 7c shows the relative positions of the wrist joint of the surgical robot arm, the surgical instrument and the pivot point in a second situation in which the surgical robotic system is not able to respect the pivot point;

FIG. 7d shows a right angled triangle whose edges represent a retraction distance (x), a perpendicular distance (y) and an actual distance (d) between the wrist joint and the pivot point; and

FIG. 8 is a graph showing an alarm threshold, a first clash threshold and a second clash threshold as functions of the retraction distance.

### DETAILED DESCRIPTION

The following description is presented to enable any person skilled in the art to make and use the invention, and is provided in the context of a particular application. Various modifications to the disclosed examples will be readily apparent to those skilled in the art. The general principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

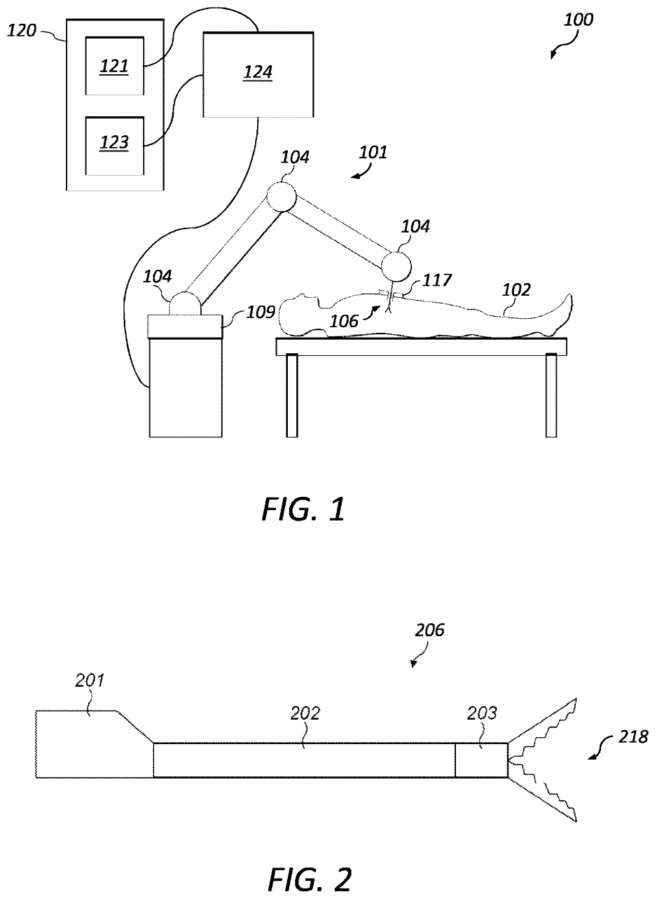

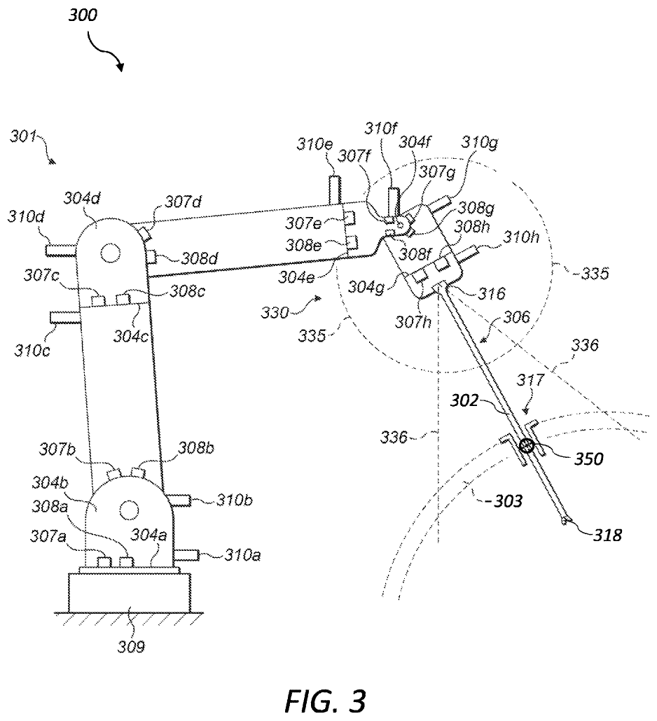

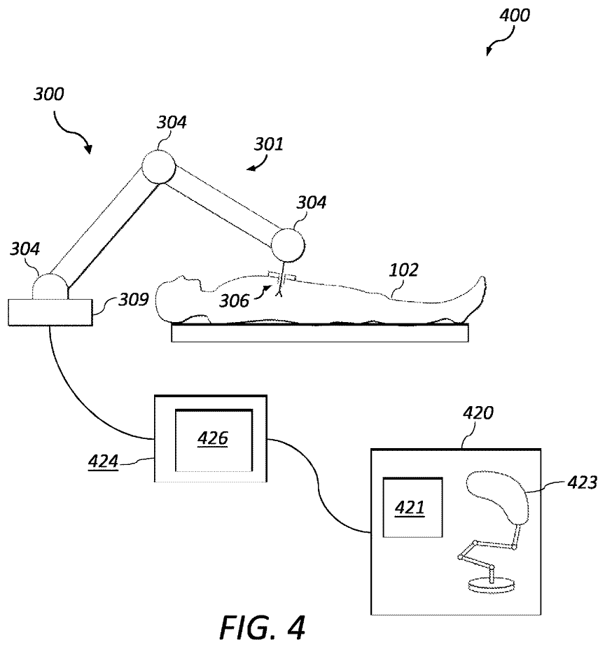

FIG. 3 shows an example of a surgical robot 300 which comprises a surgical robot arm 301 and a surgical instrument 306. The surgical robot 300 may be used within a surgical robotic system, such as the surgical robotic system 100 shown in FIG. 1, or the surgical robotic system 400 shown in FIG. 4 as will be described in further detail herein.

At its proximal end, the surgical robot arm 301 comprises a base 309. The surgical robot arm has a series of rigid arm members. Each arm member in the series is joined to the preceding arm member by a respective joint 304—shown in FIG. 3 as joints 304a-g. Joints 304a-g may be referred to as a series of joints. Joints 304a-e and 304g are revolute joints. Joint 304f is composed of two revolute joints whose axes are orthogonal to each other, e.g. as in a “Hooke's” or universal joint. Joint 304f may be termed a “wrist joint”. A surgical robot arm could be jointed differently from the surgical robot arm 301 of FIG. 3. For example, joint 304d could be omitted and/or joint 304f could permit rotation about a single axis. Alternatively, or additionally, the surgical robot arm 301 could include one or more joints that permit motion other than rotation between respective sides of the joint, such as a prismatic joint by which an instrument attachment can slide linearly with respect to more proximal parts of the surgical robot arm.

The joints are configured such that the configuration of the surgical robot arm can be altered. This allows the distal end 330 of the surgical robot arm to be moved to an arbitrary point in a three-dimensional working volume illustrated generally at 335. One way to achieve that is for the joints to have the arrangement illustrated in FIG. 3. Other combinations and configurations of joints could achieve a similar range of motion. There could be more or fewer arm members.

The distal end 330 of the surgical robot arm 301 has an attachment 316 by means of which a surgical instrument 306 can be releasably attached. Movement of the surgical robot arm 301 thereby causes movement of the surgical instrument 306. The surgical instrument 306 has a shaft 302. The surgical instrument 306 has an end effector 318 at the distal end of the shaft 302. The end effector 318 consists of a device for engaging in a procedure, for example a cutting, grasping or imaging device. As described herein, terminal joint 304g may be a revolute joint. The surgical instrument 306 and/or the attachment 316 may be configured so that the surgical instrument 306 (e.g. in particular, its shaft 302) extends linearly parallel with the rotation axis of the terminal joint 304g of the surgical robot arm 301. In this example the surgical instrument 306 (e.g. in particular, its shaft 302) extends along an axis coincident with the rotation axis of joint 304g.

For some types of minimally invasive procedure, the surgical instrument 306 may be inserted into the patient's body through a synthetic port 317. For example, the minimally invasive procedure may be performed within the patient's abdomen. The port 317 may provide a passageway through the outer tissues 303 of the patient so as to limit disruption to those tissues as the surgical instrument 306 is inserted and retracted, and as the surgical instrument 306 is moved by the surgical robot arm 301 within the patient's body. For other types of minimally invasive procedure, the surgical instrument 306 may be inserted directly into the patient's body through a natural orifice. For example, the minimally invasive procedure may be performed in the patient's throat, and the natural orifice may be the patient's mouth.

FIG. 2 shows an example surgical instrument for attachment to the surgical robot arm 301 shown in FIG. 3. The surgical instrument 206 comprises a base 201 at its proximal end by which it connects to (e.g. attaches to) the surgical robot arm 301. A shaft 202 connects the base 201 to an articulation 203. The shaft 202 is a rigid linear shaft. The articulation 203 is connected to the distal end of the shaft 202. The articulation 203 connects the shaft 202 to an end effector 218. The end effector 218 is at the distal end of the surgical instrument 206. By way of example only, in FIG. 2, a pair of serrated jaws are illustrated as the end effector 218. The articulation 203 permits the end effector 218 to move relative to the shaft 202. The skilled person would be aware of numerous articulations suitable for permitting the end effector 218 to move relative to the shaft 202, and so for conciseness the specific implementation of the articulation will not be discussed further herein.

It is to be understood that FIG. 2 shows just one specific example of a surgical instrument, and that various other suitable surgical instruments exist to which the principles described herein could be applied.

Returning to FIG. 3, joints 304e and 304f of the surgical robot arm 301 are configured so that, with the distal end 330 of the surgical robot arm 301 held at an arbitrary location in the working volume 335, the surgical instrument 306 can be moved in an arbitrary direction within a cone 336. One way to achieve that is for the terminal part of the arm to comprise the pair of joints 304e and 304f whose axes are mutually arranged as described above. Other mechanisms can achieve a similar result. For example, joint 304g could influence the attitude of the instrument if the instrument extends in a direction which is not parallel to the axis of joint 304g.

The surgical robot arm 301 comprises a series of motors 310a-h. With the exception of the compound joint 304f, which is served by two motors, each motor is arranged to drive rotation about a respective joint of the surgical robot arm 301. The motors are controlled by a control system (such as control system 124 shown in FIG. 1, or the control system 424 shown in FIG. 4 as will be described in further detail herein). The control system comprises a processor and a memory. The memory stores, in a non-transient way, software code that can be executed by the processor to cause the processor to control the motors 310a-h in order to alter the configuration of the surgical robot arm 301 in the manner described herein.

The surgical robot arm 301 may comprise a series of sensors 307a-h and 308a-h. These sensors may comprise, for each joint, a position sensor 307a-h for sensing the rotational position of the joint and a force sensor 308a-h for sensing forces (or torques) applied about the joint's rotation axis. Compound joint 304f may have two pairs of sensors. One or both of the position and force sensors for a joint may be integrated with the motor for that joint. The outputs of the sensors are passed to the control system (such as control system 124 shown in FIG. 1, or the control system 424 shown in FIG. 4 as will be described in further detail herein) where they form inputs for the processor.

It is to be understood that FIG. 3 shows just one specific example of a surgical robot arm, and that various other suitable surgical robot arms exist to which the principles described herein could be applied.

FIG. 4 shows an example a surgical robotic system 400 comprising a surgical robot arm 301 and a surgeon input device 423. The surgeon input device 423 is part of a surgeon console 420 which also comprises a display 421.

A simplified schematic of the surgical robot arm 301 is shown in FIG. 4 for ease of illustration. It is to be understood that the surgical robot arm 301 shown in FIG. 4 can have the same properties and features as the surgical robot arm 301 described with reference to FIG. 3.

In FIG. 4, the surgeon input device 423 comprises a hand controller connected to a gimbal assembly which permits the hand controller to move, e.g. with six degrees of freedom. The configuration of the gimbal assembly can be detected by sensors on the gimbal assembly and passed to the control system 424. A surgeon can move the hand controller in order to request corresponding movement of the surgical instrument 306 attached to the surgical robot arm 301. The skilled person would be aware of numerous gimbal assemblies suitable for permitting the hand controller to move with six degrees of freedom and for detecting that movement, and so for conciseness the specific implementation of the gimbal assembly will not be discussed further herein. In an alternative example, instead of the gimbal assembly, the hand controller could be equipped with accelerometers which permit its position and orientation to be estimated.

It is to be understood that FIG. 4 shows just one specific example of a surgeon input device, and that various other suitable surgeon input devices exist to which the principles described herein could be applied. For example, the surgeon input device may instead resemble a “games controller” having a plurality of joysticks that can be moved to request corresponding movement of the surgical robot arm 301.

A control system 424 is connected to the remote surgeon console 420 and to the surgical robot 300. The control system 424 may be separate from the remote surgeon console 420 and the surgical robot 300. The control system 424 may be part of the surgical robot 300. The control system 424 may be part of the remote surgeon console 420. The control system 424 may be distributed between the remote surgeon console 420 and the surgical robot 300.

The control system 424 comprises a processor and a memory. The memory stores, in a non-transient way, software code that can be executed by the processor to cause the processor to control the surgical robot arm 301 of the surgical robot 300 in the manner described herein.

The control system 424 receives inputs from the surgeon input device 423 and converts those inputs to control signals to move one or more of the joints 304 of the surgical robot arm 301 in order to alter its configuration. The control system 424 sends these control signals to the surgical robot 300, where the corresponding one or more of the joints 304 of the surgical robot arm 301 are driven accordingly. Movement of the surgical instrument 306 attached to the surgical robot arm 301 can thereby be controlled by the control system 424 in response to movement of the surgeon input device 423.

Constraints may be placed on the movement of the surgical instrument 306 that can be caused by the control system 424. One such constraint is that the control system 424 is configured to control the surgical robot arm 301, in dependence on inputs received at the surgeon input device 423, to alter the configuration of the surgical robot arm 301 whilst attempting to maintain an intersection between the surgical instrument 306 attached to the surgical robot arm 301 and a pivot point. The control system may be configured to control the surgical robot arm 301 in this way during a minimally invasive procedure. FIG. 3 shows an example pivot point 350. The pivot point may be referred to as a “fulcrum” or a “virtual pivot point”. The pivot point 350 is a point in space about which the control system 424 is configured to drive the surgical instrument 306 to pivot. In the example shown in FIG. 3, there is nothing physically present at the pivot point (which is why it may be referred to as a virtual pivot point or a virtual fulcrum) and the pivot point 350 is a software constraint enforced by the control system 424 when it determines the control signals for driving the surgical robot arm 301.

As such, the surgical robot arm 301 is driven using control signals which attempt to maintain an intersection between the surgical instrument 306 and the pivot point 350. The control signals “attempt to” maintain the intersection between the surgical instrument 306 and the pivot point 350 in the sense that they command the surgical robot arm 301 to move in a manner which would maintain the intersection between the surgical instrument 306 and the pivot point 350, but as described below it is not always possible for the surgical robot arm 301 to move exactly as commanded by the control signals so it may not always be possible to maintain the intersection between the surgical instrument 306 and the pivot point 350.

As an example, during a minimally invasive procedure, the surgeon can use the surgeon input device 423 to indicate a desired position of the surgical instrument 306 (e.g. in particular, a part of the surgical instrument 306 such as its end effector). In response, the control system 424 determines a configuration of the series of joints 304 of the surgical robot arm 301 that will result in both (i) the end effector of the surgical instrument 306 being placed in that desired position and (ii) the shaft 302 of the surgical instrument 306 passing through (e.g. maintaining an intersection with) the pivot point 350, and to generate a control signal to move the series of joints 304 to that configuration.

By determining a suitable pivot point, the disruption to the outer tissues 303 of the patient caused by moving the surgical instrument 306 during a minimally invasive procedure can be minimised. For example, a suitable pivot point may be located within the port 317, e.g. at or close to the centre of the port 317.

In a simple example, a user of the surgical robotic system 400 may determine the pivot point “by eye”. For example, prior to a minimally invasive procedure, the distal end of the shaft 302 of the surgical instrument 306 may be positioned by the user within the access port or natural orifice. When the user is satisfied that the distal end of the shaft 302 is positioned in the centre of the access port or natural orifice, they can signal to the control system 424 that the current position of the distal end of the shaft 302 should be saved as the pivot point. The control system may determine the current position of the distal end of the shaft 302 in dependence on inputs from the position sensors 307a-h that indicate the position of the attachment 316 for the surgical instrument 306 and one or more parameters of the surgical instrument 306 (e.g. including the distance between its base and the distal end of its shaft, and the orientation of its shaft relative to its base) stored in the memory of the control system or stored in a memory on the instrument itself. The control system can then store this pivot point (or “fulcrum”) in memory for later use.

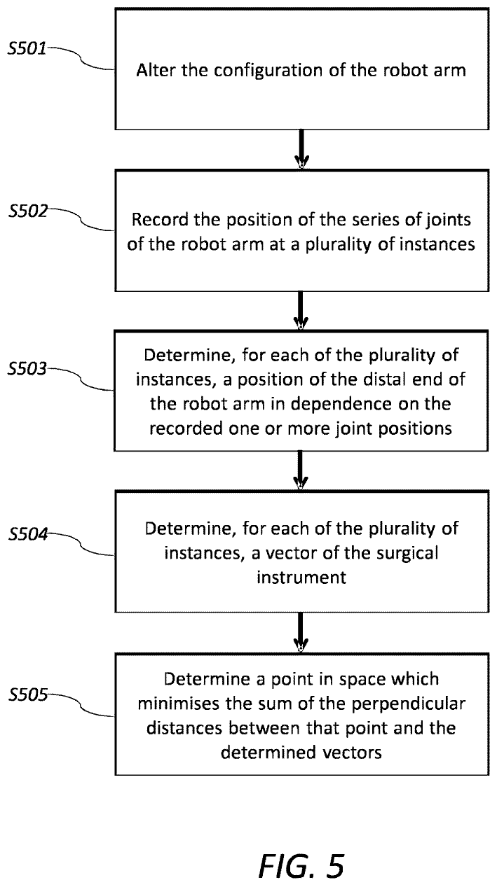

In an alternative example, a calibration process can be performed prior to performing a minimally invasive procedure in order to determine a suitable pivot point. FIG. 5 shows an example surgical robot arm calibration process in which the pivot point is determined using a port training process. The operating mode of the surgical robot 300 can be set to be a calibration mode during the calibration process so that the surgical robotic system can act accordingly in order to calibrate the surgical robot.

In step S501, the configuration of the surgical robot arm 301 can be altered whilst the surgical instrument 306 is inside the access port 317 or natural orifice.

The configuration of the surgical robot arm 301 can be altered by the application of external forces directly onto the surgical robot arm 301. For example, a member of the bedside team (e.g. an operating room nurse) may apply forces directly to the surgical robot arm 301 (e.g. by pushing a joint of the surgical robot arm 301) which can be sensed by the force sensors 308a-h and acted on by the control system 424 in a manner that would be understood by the skilled person. During the calibration process, when operating in the calibration mode, the control system 424 can control the surgical robot arm 301 to maintain a position in which it is placed by means of external forces applied directly to the surgical robot arm 301.

During the calibration process, when operating in the calibration mode, the surgical robot arm 301 can be moved generally transversely to the shaft 302 of the surgical instrument 306. The configuration of the surgical robot arm 301 may be altered such that the distal end of the surgical robot arm 301 is moved in two dimensions transverse (e.g. perpendicular) to the shaft 302: e.g. with (i) components parallel to a direction that is transverse to the shaft 302 and also with (ii) components orthogonal to that direction but transverse to the shaft 302. To do this, the operator (e.g. a member of the bedside team) may gyrate the distal end 330 of the surgical robot arm 301 about a point generally aligned with the natural axis of the access port or natural orifice. This causes the surgical instrument 306 to come into contact with the access port 317 (or natural orifice) such that the access port 317 (or natural orifice) applies a lateral force on the shaft 302. That force can be accommodated by motion about the joint 304f. The force is “lateral” in the sense that it is applied to the sides of the instrument and is generally in a direction that is transverse (e.g. perpendicular) to the shaft 302 of the instrument 306.

In step S502, as the configuration of the surgical robot arm 301 is being altered, the position sensors 307a-h can record the position of each joint 304 of the series of joints of the surgical robot arm 301. The position sensors 307a-h can record the positions of each joint of the surgical robot arm 301 at a plurality of instances in time. Position information may be recorded irregularly or at predetermined intervals, e.g. every 20 milliseconds (i.e. at a frequency of 50 Hz). The position sensors provide the recorded position information to the control system 424. The control system may also store in memory information indicating one or more parameters of the surgical instrument 306 (e.g. including the length of the shaft and/or the orientation of its shaft relative to its base). In some examples, these parameters of the surgical instrument 306 may be read from a memory on the surgical instrument itself and passed to the control system 424.

In steps S503 and S504, the control system 424 uses this information to determine, at each of the plurality of instances in time: (a) the position of the distal end 330 of the surgical robot arm 301 relative to the base 309 and (b) a vector representing the surgical instrument 306 (e.g. in particular, its shaft 302) relative to the distal end 330 of the surgical robot arm 301. Position (a) and vector (b) may be termed a data pair.

The vectors of the data pairs will approximately (but usually not exactly) converge, from their respective distal end position, on the natural rotation centre of the access port 317 or natural orifice. By collecting a plurality of said data pairs, and then solving for a best estimate (i.e. an estimate with the least error) of a location where the vectors converge, the control system 424 can determine a fulcrum (e.g. a pivot point) within the access port or natural orifice. For example, in step S505, the control system 424 may estimate, as the pivot point, the point in space which minimises the sum of the perpendicular distances between that point and the vectors of the data pairs. Here, a “perpendicular distance” between a point and a vector refers to the distance between the point and the vector in a direction perpendicular to the vector. Therefore, the perpendicular distance between a point and a vector is the distance between the point and the position on the vector which is closest to the point. In some examples, the control system 424 may estimate, as the pivot point, the point in space which minimises the sum of the squares of the perpendicular distances between that point and the vectors of the data pairs. Methods for finding a best estimate by minimising a sum of differences or by minimising a sum of squared differences are known in the art. The control system 424 can store the pivot point in memory for later use.

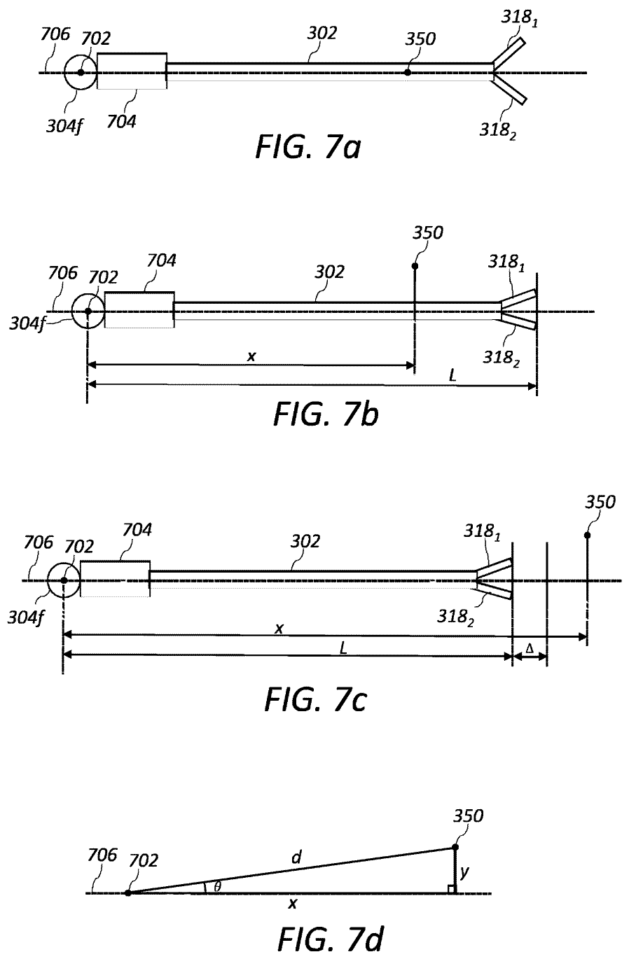

As described above, the control system 424 determines control signals which command the surgical robot arm 301 to move so as to respect the pivot point (i.e. so that the instrument shaft intersects the pivot point. FIG. 7a illustrates the relative positions of a wrist joint 304f of the surgical robot arm 302, the surgical instrument and a pivot point 350 when the surgical robotic system 100 is operating normally. The centre of the wrist joint is denoted 702 in FIG. 7a. As described above, the wrist joint 304f is the most distal joint of the joints of the surgical robot arm 301 which control the orientation of the surgical instrument. As described above, the surgical instrument comprises a shaft 302 and an end effector 318, which in this example comprises a set of jaws with two end effector elements 3181 and 3182. The surgical robot comprises one or more other components 704 in between the wrist joint 304f and the shaft of the instrument 302. These other components 704 may include another rotational joint 304g (which will not affect the orientation of the instrument) and/or an interface by which the instrument 306 is attached to the surgical robot arm 301. FIG. 7a also shows a shaft axis line 706 which is coincident with an axis of the shaft 302 of the surgical instrument and extends beyond the ends of the shaft 302. In the situation shown in FIG. 7a the surgical robotic system 400 is respecting the pivot point, i.e. the shaft axis line 706 intersects the pivot point 350.

However, sometimes the surgical robot arm 301 does not move exactly as commanded by the control signals. This may be because other forces are applied to the surgical robot (e.g. by a user, such as a member of the bedside team) and the surgical robot arm 301 is flexible. In particular a drivetrain of the surgical robot arm 301 is flexible. The drivetrain couples a motor input to a joint output. Since the drivetrain is flexible there may be a difference between the position of the motor input and the output of the joint. As such, sometimes the actual configuration of the surgical robot arm 301 is not the same as the commanded configuration, such that sometimes the surgical robotic system 400 is not able to respect the pivot point, i.e. sometimes the shaft axis line 706 does not intersect the pivot point 350.

A first situation in which the surgical robotic system 400 is not able to respect the pivot point 350 is shown in FIG. 7b. In this case it can be seen that shaft axis line 706 does not intersect the pivot point 350. In other words, the perpendicular distance between the shaft axis line 706 and the pivot point 350 is non-zero. The “perpendicular distance” between the shaft axis line 706 and the pivot point 350 is the distance between the shaft axis line 706 and the pivot point 350 in a direction perpendicular to the shaft axis line 706. Therefore, the perpendicular distance between the shaft axis line 706 and the pivot point 350 is the distance between the pivot point 350 and the position on the shaft axis line 706 which is closest to the pivot point 350. When the surgical robotic system 400 is able to respect the pivot point then the perpendicular distance between the shaft axis line 706 and the pivot point 350 is zero (e.g. as shown in FIG. 7a), but when the surgical robotic system 400 is not able to respect the pivot point then the perpendicular distance between the shaft axis line 706 and the pivot point 350 may be non-zero (e.g. as shown in FIGS. 7b and 7c).

If the perpendicular distance between the shaft axis line 706 and the pivot point 350 becomes too large then it could cause a risk to the safety of the patient, and/or it may be a sign that the surgical robotic system 400 is not operating correctly. As such, an alarm system 426 is implemented in the surgical robotic system 400 to raise an alarm if it detects that the perpendicular distance between the shaft axis line 706 and the pivot point 350 is above an alarm threshold. FIG. 4 shows the alarm system 426 being implemented as part of the control system 424, but in other examples the alarm system 426 could be implemented within the surgical robotic system 400 somewhere other than in the control system 424, e.g. it could be implemented in the surgical robot 300. The alarm system 426 could be implemented in software, e.g. by executing instructions of a computer program on one or more processors (e.g. on one or more processors of the control system 424). The instructions could be stored in a computer readable medium, e.g. in a memory of the control system 424.

In a simplistic example, the alarm system 426 could compare the perpendicular distance between the shaft axis line 706 and the pivot point 350 with a constant threshold to determine whether to raise an alarm (e.g. a medium priority alarm). For example the constant threshold could be 0.025 m. However, using a constant threshold means that the alarm system is more sensitive to perturbations of the pose of the wrist joint 304f when the surgical instrument is retracted such that the wrist joint 304f is further away from the pivot point 350 compared to when the surgical instrument is inserted such that the wrist joint 304f is closer to the pivot point 350. This can be seen with reference to FIG. 7d which shows a right angled triangle whose edges represent a retraction distance (x), a perpendicular distance (y) and an actual distance (d) between the centre of the wrist joint 702 and the pivot point 350. The “retraction distance” is the component of the distance between the centre of the wrist joint 702 and the pivot point 350 in a direction parallel to the shaft axis line 706. As such, the retraction distance may be referred to as the “pivot point along-shaft distance”. The perpendicular distance is the component of the distance between the centre of the wrist joint 702 and the pivot point 350 in a direction perpendicular to the shaft axis line 706, and may be referred to as the “pivot point off-shaft distance”. The angle between the shaft axis line and the line connecting the centre of the wrist joint 702 and the pivot point 350 is controlled by the pose of the wrist joint 304f and is denoted as e in FIG. 7d. The “pose” of the wrist joint 304f comprises the position (x,y,z) and orientation (pitch, yaw, roll) of the wrist joint 304f. It can be appreciated that for a given perturbation to the orientation of the wrist joint, i.e. for a given value of 0, the perpendicular distance, y, will be larger if the retraction distance, x, is larger. Therefore, since it is the orientation of the wrist joint 304f that controls the orientation of the instrument, and since it is the perpendicular distance, y, that is compared with the alarm threshold by the alarm system 426 then having a constant alarm threshold means that the alarm system 426 is more sensitive to perturbations of the orientation of the wrist joint 304f when the surgical instrument is retracted such that the wrist joint 304f is further away from the pivot point 350 compared to when the surgical instrument is inserted such that the wrist joint 304f is closer to the pivot point 350.

The control system 424 uses the perpendicular distance, y, to try to ensure that the actual wrist pose (e.g. according to measurements taken by the position sensors 307a-h) is consistent with the pivot point 350. The control system 424 tries to make the actual wrist pose match the commanded wrist pose, so that transient perturbations to the wrist pose (which cause the perpendicular distance, y, to be non-zero can be quickly and automatically corrected.

In the simplistic example of the alarm system 426 given above, the extra sensitivity of the alarm system 426 when the surgical instrument is retracted away from the pivot point 350 can result in the alarm being raised unnecessarily. In particular, it is not useful to have a check on the wrist pose which is at its tightest (i.e. which allows the smallest perturbation before an alarm is raised) when the surgical instrument 306 is fully retracted from the port 117, i.e. fully retracted outside of the patient 102. As described above, raising an alarm causes a significant disruption to the workflow of a surgical procedure. Some functionality may be disabled and the surgical robotic system may need to be reset or retrained after an alarm has been raised before the workflow of the surgical procedure can continue. As such, avoiding raising an alarm in situations when it is not necessary for an alarm to be raised will improve the workflow of the surgical procedure.

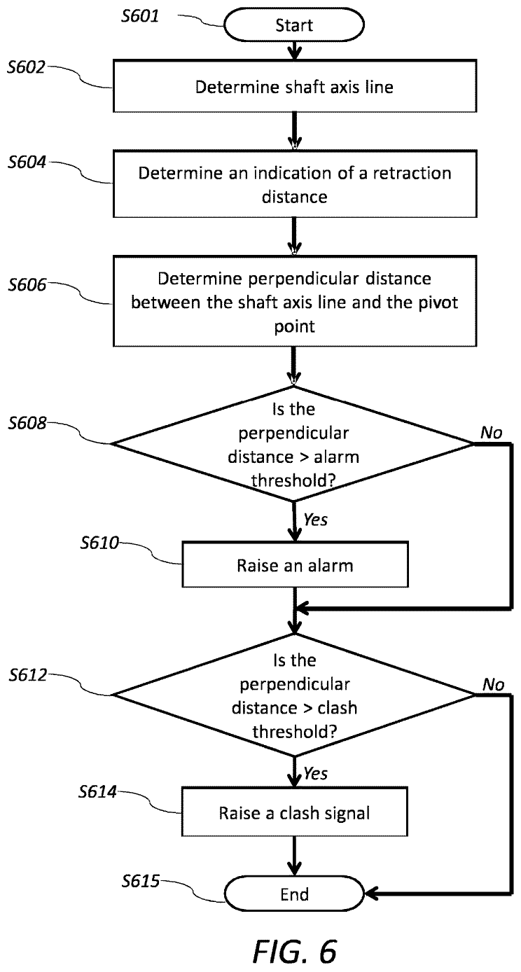

An improved method of raising an alarm for the surgical robot 300 is described with reference to the flow chart of FIG. 6. The method starts at S601.

In step S602 the alarm system 426 determines the shaft axis line 706. As described above, the shaft axis line 706 is coincident with an axis of the shaft 302 of the instrument 306. The alarm system 426 may “determine” the shaft axis line 706 in step S602 by calculating the shaft axis line 706 or by receiving an indication of the shaft axis line 706 which has been calculated somewhere other than the alarm system 426. It is noted that the shaft axis line 706 is an extension of the roll axis of joint 304g. The control system 424 may calculate the shaft axis line 706 (or the roll axis of joint 304g) as part its process of determining the control signals for controlling the surgical robot arm 301. Processes for the determining the control signals in the control system 424 are beyond the scope of this disclosure but would be known to a person skilled in the art.

In step S604 the alarm system 426 determines an indication of a retraction distance, x, which indicates how far the instrument is retracted relative to the pivot point. The alarm system 426 may “determine” the indication of the retraction distance, x, in step S604 by calculating the indication of the retraction distance or by receiving the indication of the retraction distance which has been calculated somewhere other than the alarm system 426. As mentioned above, the retraction distance, x, is the component of the distance between the centre of the wrist joint 702 and the pivot point 350 in a direction parallel to the shaft axis line 706. An indication of the retraction distance may be calculated by the control system 424 as part of its process of determining the control signals for controlling the surgical robot arm 301. The indication of the retraction distance could be determined by determining a distance along the shaft axis line 706 between a predetermined position on the shaft axis line 706 and the point on the shaft axis line 706 which is closest to the pivot point 350. For example, the predetermined position on the shaft axis line may be point 702 which is at the most distal joint (i.e. the wrist joint 304f) of the joints of the surgical robot arm 301 which control the orientation of the shaft axis line 706.

In some examples, in step S604, an insertion distance could be determined which indicates how far the instrument is inserted relative to the pivot point. An insertion distance, could be calculated as L−x, where L is the distance from the centre of the wrist joint 702 to the tip of the instrument 306, such that determining the insertion distance would provide an indication of the retraction distance, x. It is noted that the distance, L, can be easily determined using instrument parameters stored in the memory of the control system 424 or on the instrument itself.

In step S606 the alarm system 426 determines the perpendicular distance, y, between the shaft axis line 706 and the pivot point 350 in a direction perpendicular to the shaft axis line 706. The alarm system 426 may “determine” the indication of the perpendicular distance, y, in step S606 by calculating the perpendicular distance or by receiving an indication of the perpendicular distance which has been calculated somewhere other than the alarm system 426. The retraction distance, x, is known (from step S604) and the angle θ can be determined by comparing the actual pose of the wrist joint and the commanded pose of the wrist joint which would make the shaft axis line 706 intersect the pivot point 350. So the perpendicular distance, y, can be calculated as y=x tan θ. It is noted that the angle θ is in two dimensions (pitch and yaw). Furthermore, it is noted that the measured wrist cartesian position (i.e. x,y,z position) may not correspond with the desired wrist position, and the measured wrist orientation (i.e. pitch, yaw, roll) may not correspond with the desired wrist orientation.

In step S608 the alarm system 426 compares the determined perpendicular distance, y, with an alarm threshold. If the perpendicular distance, y, is greater than the alarm threshold then the method passes to step S610. If the perpendicular distance, y, is less than the alarm threshold then the method skips step S610, i.e. step S610 is not performed and the method passes straight to step S612. In the example shown in FIG. 6 a “greater than” comparison is performed in step S608 such that if the perpendicular distance equals the alarm threshold then step S610 is not performed, but in other examples a “greater than or equals” comparison may be performed in step S608 such that if the perpendicular distance equals the alarm threshold then step S610 would be performed.

In step S610 the alarm system 426 raises an alarm. As described above, the alarm may be a medium priority alarm, and will cause a disruption to the workflow of the surgical procedure. The alarm system 426 may raise an alarm in a number of different ways. For example, the alarm system 426 may raise the alarm by doing one or more of the following: (i) switch on a warning light (e.g. on the display 421 of the surgeon console 420), (ii) output an audible alarm (e.g. on the surgeon console 420), (iii) disable one, some or all functions of the surgical robot 300, (iv) change an operating mode of the surgical robot arm 301, (v) causing the hand controllers to vibrate, and (vi) show an alarm sign in the surgeon display and/or another display in the Operating Room. For example, the operating mode of the surgical robot arm 301 may be changed to a “faulted locked” mode, which is a special mode for when a fault occurs in which the surgical robot has limited functionality.

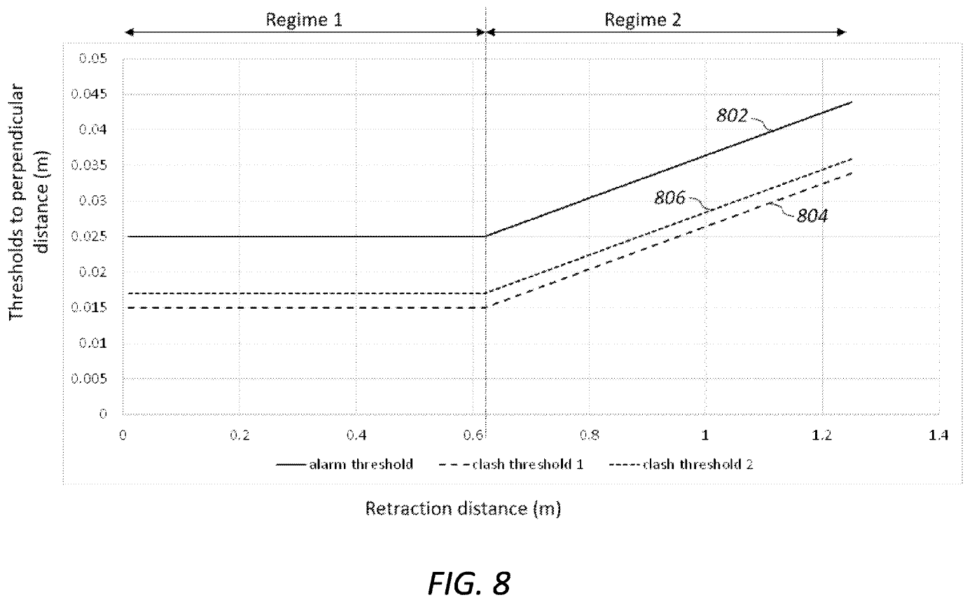

In contrast to the simplistic example described above in which the alarm threshold is a constant value, in examples described herein the alarm threshold is dependent upon the indicated retraction distance, x. This allows the alarm threshold to have a suitable value which can change as the instrument is retracted or inserted relative to the pivot point 350. For example, the alarm threshold may be a function of the determined indication of the retraction distance, x. FIG. 8 is a graph showing an alarm threshold 802 as a function of the retraction distance, x. As shown in FIG. 8, the alarm threshold 802 has two regimes: (i) a first regime (denoted “Regime 1” in FIG. 8) for a first range of retraction distances, and (ii) a second regime (denoted “Regime 2” in FIG. 8) for a second range of retraction distances. The first and second ranges of retraction distances are non-overlapping but they are contiguous (i.e. there is not a gap between the first and second ranges of retraction distances). In the example shown in FIG. 8 the first range of retraction distances is below 0.62 m and the second range of retraction distances is 0.62 m and above.

In the first range of retraction distances, the tip of the instrument is either at least as far along the shaft axis line 706 as the pivot point 350 or less far along the shaft axis line 706 than the pivot point 350 by an amount which is not greater than a predetermined amount (A). Here ‘further along the shaft axis line 706’ would mean further from the wrist join 304f, i.e. further to the right in FIGS. 7a, 7b and 7c. FIG. 7b shows an example in which the tip of the instrument is further along the shaft axis line 706 than the pivot point 350 (i.e. the tip of the instrument is to the right of the pivot point 350 in FIG. 7b). As can be seen in FIG. 3, the pivot point 350 is often positioned within the port 317 which is in the outer tissue 303 of the patient 102. As such, when the tip of the instrument is further along the shaft axis line 706 than the pivot point 350 then the tip of the instrument can be considered to be inside the patient 102. Regime 1 is used for the alarm threshold when at least some of the surgical instrument is inside the patient 102. In the example shown in FIG. 8, the alarm threshold has a constant value (Cₐₗₐᵣₘ) in the first regime, which in this example is 0.025 m. In other examples, the constant value could be different. Furthermore, in some other examples, the alarm threshold might not have a constant value in regime 1. By setting the alarm threshold for regime 1 to be a constant value (in particular the same constant value that is used in the simplistic example described above in which the alarm threshold is not dependent upon the retraction distance at all) the improved alarm system will raise an alarm in the same situations as in the simplistic example whilst any part of the instrument is within the patient or is within a small distance (e.g. 4 cm) from the patient. As such, patient safety is ensured to be as safe when using the improved alarm system as when using the simplistic alarm system.

In the second range of retraction distances, the tip of the instrument is less far along the shaft axis line 706 than the pivot point 350 by an amount which is greater than the predetermined amount (Δ). Here “less far along” means closer to the wrist joint 304f, i.e. further to the left in FIGS. 7a, 7b and 7c. FIG. 7c shows another example in which the surgical robotic system 400 is not able to respect the pivot point 350. In the example shown in FIG. 7c the tip of the instrument is less far along the shaft axis line 706 than the pivot point 350 by an amount which is greater than the predetermined amount (denoted A in FIG. 7c), i.e. the tip of the instrument is more than A to the left of the pivot point 350 in FIG. 7c. When the tip of the instrument is less far along the shaft axis line 706 than the pivot point 350 by an amount which is greater than the predetermined amount (Δ) then it is safe to assume that no part of the surgical robot 301 (which includes the surgical instrument 306) is inside the patient 102. The predetermined distance A is used as a safety buffer to ensure that this assumption errs on the side of caution, i.e. to ensure that the Regime 2 is never used when any part of the surgical robot (which includes the surgical instrument) is inside the patient. As an example, A may be 4 cm, but in other examples A may have a different value. In this way, regime 2 may be (and can only be) used for the alarm threshold when none of the surgical instrument is inside the patient 102. In the example shown in FIG. 8, the alarm threshold has a value in the second regime that increases as the indicated retraction distance, x, increases. In particular, in the example shown in FIG. 8, the alarm threshold has a value in the second regime that increases linearly as the indicated retraction distance, x, increases. In this way, the alarm threshold is relaxed as a linear function of the retraction distance, x, when the surgical instrument is outside of the patient 102.

The alarm threshold is a continuous function of the determined indication of the retraction distance. So where the two regimes meet (e.g. at a retraction distance of 0.62 m in the example shown in FIG. 8) the functions for the two regimes have the same value. This avoids a discontinuity in the alarm threshold.

For example, the alarm threshold (Tₐₗₐᵣₘ) 802 can be given as:

$${T}_{alarm} = {C}_{alarm} + K max ( 0 , x - ( L + Δ ) )$$

where Cₐₗₐᵣₘ is the constant value that the alarm threshold has in the first regime; K represents the gradient of the alarm threshold in regime 2 as shown in FIG. 8; x is the retraction distance, i.e. the component of the distance between the centre of the wrist joint 702 and the pivot point 350 in a direction along the shaft axis line 706; L is the distance between the centre of the wrist joint 702 and the tip of the instrument; and A is a predetermined distance. In regime 1, x−(L+Δ)≤0 so Tₐₗₐᵣₘ=Cₐₗₐᵣₘ. In regime 2, x−(L+Δ)≥0 so Tₐₗₐᵣₘ=Cₐₗₐᵣₘ+K(x−(L+Δ)). To give some example values, Cₐₗₐᵣₘ could be 0.025 m, K could be 0.03, L could be 0.58 m and A could be 0.04 m, but in other examples these parameters could have different values. In the examples shown in FIGS. 7b and 7c, L is the distance between the centre of the wrist joint 702 and the tip of the instrument, but in other examples L could be something different, e.g. it could be the distance between the centre of the wrist joint 702 and the axis of the jaws of the instrument (such that it wouldn't include the length of the jaws). In these other examples, the value of A may be increased to provide more of a safety buffer.

By increasing the alarm threshold in regime 2 from that in regime 1 the improved alarm system will be less likely to raise an alarm than in the simplistic example (in which the alarm threshold is not dependent upon the retraction distance at all) when it is known that no part of the instrument is within the patient 102. As such, patient safety is not compromised but the number of times that the alarm may be raised unnecessarily will be reduced. It is noted that in the first regime Tₐₗₐᵣₘ=Cₐₗₐᵣₘ and in the second regime Tₐₗₐᵣₘ≥Cₐₗₐᵣₘ so the number of alarms that are raised in the improved alarm system will be less than or equal to the number of alarms that would be raised in the simplistic alarm system (in which the alarm threshold is not dependent upon the retraction distance at all).

In step S612 the alarm system 426 compares the determined perpendicular distance, y, with a clash threshold. If the perpendicular distance, y, is greater than the clash threshold then the method passes to step S614. If the perpendicular distance, y, is less than the clash threshold then the method skips step S614, i.e. step S614 is not performed and the method passes straight to step S615. The clash threshold is less than the alarm threshold for all retraction distances. The difference between the clash threshold and the alarm threshold may be constant for all retraction distances. In the example shown in FIG. 6 a “greater than” comparison is performed in step S612 such that if the perpendicular distance equals the clash threshold then step S614 is not performed, but in other examples a “greater than or equals” comparison may be performed in step S612 such that if the perpendicular distance equals the clash threshold then step S614 would be performed.

In step S614 the alarm system 426 raises a clash signal. The clash signal will prompt a user to take corrective action in situations when the perpendicular distance, y, is close to (but below) the alarm threshold. Warning the user that the perpendicular distance is getting close to the alarm threshold should reduce the number of times that the alarm needs to be raised. Raising a clash signal may slightly disrupt the workflow of the procedure, but the disruption caused by raising a clash signal is not as significant as the disruption caused by raising the alarm. The alarm system 426 may raise a clash signal in a number of different ways. For example, the alarm system 426 may raise the clash signal by doing one or more of the following: (i) display a clash warning icon on a screen (e.g. on the display 421 of the surgeon console 420 or on another display in the Operating Room), (ii) output an audible warning, (iii) cause the hand controller 423 to vibrate, iv) disable one, some or all functions of the surgical robot 300, and (v) change an operating mode of the surgical robot arm 301.

The clash threshold is dependent upon the indicated retraction distance, x. Furthermore, the clash threshold may be dependent upon the operating mode of the surgical robot arm. FIG. 8 shows two clash thresholds denoted “clash threshold 1” 804 and “clash threshold 2” 806, which are both functions of the retraction distance, x. The second clash threshold may be used in an “instrument adjust” or an “instrument change” operating mode, whilst the first clash threshold may be used in other operating modes. As shown in FIG. 8, both the first and second clash signals 804 and 806 have two regimes: (i) a first regime (denoted “Regime 1” in FIG. 8) for a first range of retraction distances, and (ii) a second regime (denoted “Regime 2” in FIG. 8) for a second range of retraction distances. These regimes are the same as described above in relation to the alarm threshold. In particular, the first and second ranges of retraction distances are non-overlapping but they are contiguous (i.e. there is not a gap between the first and second ranges of retraction distances). In the example shown in FIG. 8 the first range of retraction distances is below 0.62 m and the second range of retraction distances is 0.62 m and above. In other examples there may be only a single clash threshold which is used for all operating modes of the surgical robot arm.

As described above, in the first range of retraction distances, the tip of the instrument is either at least as far along the shaft axis line 706 as the pivot point 350 (e.g. as shown in FIG. 7b) or less far along the shaft axis line than the pivot point by an amount which is not greater than a predetermined amount (Δ). As described above, when the tip of the instrument is further along the shaft axis line 706 than the pivot point 350 then the tip of the instrument is inside the patient 102. Regime 1 is used for the clash thresholds when at least some of the surgical instrument is inside the patient 102. In the example shown in FIG. 8, the first clash threshold has a constant value (C_{clash,1}) in the first regime, which in this example is 0.015 m, and the second clash threshold has a constant value (C_{clash,2}) in the first regime, which in this example is 0.017 m. In other examples, the constant values could be different. Furthermore, in some other examples, the clash threshold(s) might not be constant in regime 1.

In the second range of retraction distances, the tip of the instrument is less far along the shaft axis line 706 by an amount which is greater than the predetermined amount (Δ), as shown in FIG. 7c. As described above, when the tip of the instrument is less far along the shaft axis line 706 than the pivot point 350 by an amount which is greater than the predetermined amount (Δ) then it is safe to assume that no part of the surgical robot 301 (which includes the surgical instrument 306) is inside the patient 102. As an example, A may be 4 cm, but in other examples A may have a different value. In this way, regime 2 may be (and can only be) used for the clash threshold(s) when none of the surgical instrument is inside the patient 102. In the example shown in FIG. 8, the first and second clash thresholds each have a value in the second regime that increases as the indicated retraction distance, x, increases. In particular, in the example shown in FIG. 8, the clash thresholds each have a value in the second regime that increases linearly as the indicated retraction distance, x, increases. In this way, each of the clash thresholds is relaxed as a linear function of the retraction distance, x, when the surgical instrument is outside of the patient 102.

The difference between each of the clash thresholds and the alarm threshold is constant. For example, the difference between the first clash threshold and the alarm threshold is 0.01 m. The difference between the second clash threshold and the alarm threshold is 0.008 m. When the difference between a clash threshold and the alarm threshold is larger, a user will be given more warning that the perpendicular distance is getting close to the alarm threshold. This may be considered to be beneficial because it provides more opportunity for the user to take corrective action to avoid the alarm being raised, but it may also be considered to be detrimental because it means that the clash signal will be raised more often. So there is a balance to be considered when setting the difference between the clash threshold and the alarm threshold, and this balance may be different for different operating modes, which is why the clash threshold can be set to be different in different operating modes.

Each of the clash thresholds is a continuous function of the determined indication of the retraction distance. So where the two regimes meet (e.g. at a retraction distance of 0.62 m in the example shown in FIG. 8) the functions for the two regimes have the same value. This avoids a discontinuity in the clash threshold.

For example, the first clash threshold (T_{clash,1}) 804 and the second clash threshold (T_{clash,2}) 806 can be given as:

$$\begin{matrix}{T}_{clash , 1} = {C}_{clash , 1} + K max ( 0 , x - ( L + Δ ) ) \\ {T}_{clash , 2} = {C}_{clash , 2} + K max ( 0 , x - ( L + Δ ) )\end{matrix}$$

where C_{clash,1} is the constant value that the first clash threshold 804 has in the first regime; C_{clash,2} is the constant value that the second clash threshold 806 has in the first regime; K represents the gradient of the first and second clash thresholds 804 and 806 in regime 2 (which is the same as the gradient of the alarm threshold 802 in regime 2) as shown in FIG. 8; x is the retraction distance, i.e. the component of the distance between the centre of the wrist joint 702 and the pivot point 350 in a direction along the shaft axis line 706; L is the distance between the centre of the wrist joint 702 and the tip of the instrument; and A is a predetermined distance. In regime 1, x−(L+Δ)≤0 so T_{clash,1}=C_{clash,1} and T_{clash,2}=C_{clash,2}. In regime 2, x−(L+Δ)≥0 so T_{clash,1}=C_{clash,1}+K(x−(L+A) and T_{clash,2}=C_{clash,2}+K(x−(L+A). To give some example values, C_{clash,1} could be 0.015 m, C_{clash,2} could be 0.017 m, K could be 0.03, L could be 0.58 m and A could be 0.04 m, but in other examples these parameters could have different values. As mentioned above, in the examples shown in FIGS. 7b and 7c, L is the distance between the centre of the wrist joint 702 and the tip of the instrument, but in other examples L could be something different, e.g. it could be the distance between the centre of the wrist joint 702 and the axis of the jaws of the instrument (such that it wouldn't include the length of the jaws). In these other examples, the value of A may be increased to provide more of a safety buffer.

Following step S614 the method passes to step S615. In step S615 the method ends. The method shown in FIG. 6 can be performed repeatedly, e.g. at regular intervals, e.g. once every 0.2 milliseconds (i.e. at a frequency of 5 kHz).

The method shown in FIG. 6 shows the use of both an alarm threshold which is dependent upon the retraction distance for raising an alarm and a clash threshold which is dependent upon the retraction distance for raising a clash signal. In some examples, just one of these may be implemented, e.g. just the alarm threshold which is dependent upon the retraction distance may be implemented (such that steps S612 and S614 are not performed), or just the clash threshold which is dependent upon the retraction distance may be implemented (such that steps S608 and S610 are not performed, and step S604 may be optional).

If only the clash threshold is dependent upon the retraction distance, then examples described herein can be considered to provide a clash detection system for a surgical robot, wherein the clash detection system is configured to: (i) determine a shaft axis line 706 which is coincident with an axis of a shaft 302 of the instrument; (ii) determine a perpendicular distance, y, between the shaft axis line 706 and the pivot point 350 in a direction perpendicular to the shaft axis line 706; and (iii) compare the determined perpendicular distance, y, with a clash threshold and raise a clash signal in response to determining that the determined perpendicular, y, distance is greater than the clash threshold. The clash detection system could also determine an indication of a retraction distance, x, which indicates how far the instrument is retracted relative to the pivot point 350, wherein the clash threshold may be dependent upon the indicated retraction distance, x.

The robot arm described herein could be for purposes other than surgery. For example, the port could be an inspection port in a manufactured article such as a car engine and the robot arm could control a viewing instrument for viewing inside the engine.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. An alarm system of a surgical robot, the surgical robot comprising a surgical robot arm and a surgical instrument attached to a distal end of the surgical robot arm, wherein the surgical robot arm is driven using control signals which attempt to maintain an intersection between the surgical instrument and a pivot point, wherein the alarm system is configured to:
determine a shaft axis line which is coincident with an axis of a shaft of the instrument;
determine an indication of a retraction distance which indicates how far the instrument is retracted relative to the pivot point;
determine a perpendicular distance between the shaft axis line and the pivot point in a direction perpendicular to the shaft axis line; and
compare the determined perpendicular distance with an alarm threshold and raise an alarm in response to determining that the determined perpendicular distance is greater than the alarm threshold,
wherein the alarm threshold is dependent upon the indicated retraction distance.

2. The alarm system of claim 1 wherein the alarm system is configured to determine the indication of the retraction distance by determining a distance along the shaft axis line between a predetermined position on the shaft axis line and the point on the shaft axis line which is closest to the pivot point.

3. The alarm system of claim 2 wherein the surgical robot arm comprises a series of joints by which its configuration can be altered, and wherein the predetermined position on the shaft axis line is at the most distal joint of the joints which control the orientation of the shaft axis line.

4. The alarm system of claim 1 wherein the alarm threshold is a function of the determined indication of the retraction distance and has two regimes: (i) a first regime for a first range of retraction distances in which the tip of the instrument is either at least as far along the shaft axis line as the pivot point or less far along the shaft axis line than the pivot point by an amount which is not greater than a predetermined amount, and (ii) a second regime for a second range of retraction distances in which the tip of the instrument is less far along the shaft axis than the pivot point by an amount which is greater than the predetermined amount.

5. The alarm system of claim 4 wherein the alarm threshold has a constant value in the first regime.

6. The alarm system of claim 4 or 5 wherein the alarm threshold has a value in the second regime that increases as the indicated retraction distance increases.

7. The alarm system of claim 6 wherein the alarm threshold has a value in the second regime that increases linearly as the indicated retraction distance increases.

8. The alarm system of claim 4 wherein the alarm threshold is a continuous function of the determined indication of the retraction distance.

9. The alarm system of claim 1 wherein the alarm system is configured to raise the alarm by doing one or more of the following: (i) switch on a warning light, (ii) output an audible alarm, (iii) disable one, some or all functions of the surgical robot, (iv) change an operating mode of the surgical robot arm, (v) cause one or more hand controllers to vibrate, and (vi) display an alarm sign.

10. The alarm system of claim 1 wherein the alarm system is configured to not raise the alarm in response to determining that the perpendicular distance is less than the alarm threshold.

11. The alarm system of claim 1 wherein the pivot point is determined using a port training process.

12. The alarm system of claim 1 wherein the alarm system is further configured to compare the determined perpendicular distance with a clash threshold and raise a clash signal in response to determining that the determined perpendicular distance is greater than the clash threshold.

13. The alarm system of claim 12 wherein the clash threshold is dependent upon the indicated retraction distance.

14. (canceled)

15. The alarm system of claim 12 wherein the clash threshold is less than the alarm threshold.

16. The alarm system of claim 12 wherein the clash threshold is a function of the determined indication of the retraction distance and has two regimes: (i) a first regime for a first range of retraction distances in which the tip of the instrument is either at least as far along the shaft axis line as the pivot point or less far along the shaft axis line than the pivot point by an amount which is not greater than a predetermined amount, and (ii) a second regime for a second range of retraction distances in which the tip of the instrument is less far along the shaft axis than the pivot point by an amount which is greater than the predetermined amount.

17. The alarm system of claim 16 wherein the clash threshold has a constant value in the first regime.

18. The alarm system of claim 16 or 17 wherein the clash threshold has a value in the second regime that increases as the indicated retraction distance increases.

19. (canceled)

20. (canceled)

21. The alarm system of claim 12 wherein the alarm system is configured to raise the clash signal by doing one or more of the following: (i) display a clash warning icon on a screen, (ii) output an audible warning, (iii) cause a hand controller of a surgeon console to vibrate, (iv) disable one, some or all functions of the surgical robot 300, and (v) change an operating mode of the surgical robot arm 301.

22. (canceled)

23. (canceled)

24. A computer readable storage medium have stored thereon computer readable instructions that when executed on one or more processors cause a method of raising an alarm for a surgical robot to be performed, the surgical robot comprising a surgical robot arm and a surgical instrument attached to a distal end of the surgical robot arm, wherein the surgical robot arm is driven using control signals which attempt to maintain an intersection between the surgical instrument and a pivot point, wherein the method of raising an alarm comprises:
determining a shaft axis line which is coincident with an axis of a shaft of the instrument;
determining an indication of a retraction distance which indicates how far the instrument is retracted relative to the pivot point;
determining a perpendicular distance between the shaft axis line and the pivot point in a direction perpendicular to the shaft axis line;
comparing the determined perpendicular distance with an alarm threshold; and
raising an alarm in response to determining that the determined perpendicular distance is greater than the alarm threshold,
wherein the alarm threshold is dependent upon the indicated retraction distance.

25. A method of raising an alarm for a surgical robot, the surgical robot comprising a surgical robot arm and a surgical instrument attached to a distal end of the surgical robot arm, wherein the surgical robot arm is driven using control signals which attempt to maintain an intersection between the surgical instrument and a pivot point, wherein the method comprises:
determining a shaft axis line which is coincident with an axis of a shaft of the instrument;
determining an indication of a retraction distance which indicates how far the instrument is retracted relative to the pivot point;
determining a perpendicular distance between the shaft axis line and the pivot point in a direction perpendicular to the shaft axis line;
comparing the determined perpendicular distance with an alarm threshold; and
raising an alarm in response to determining that the determined perpendicular distance is greater than the alarm threshold,
wherein the alarm threshold is dependent upon the indicated retraction distance.
